(19) United States
(12) Patent Application Publication
(10) Pub. No.: US 2025/0362275 A1
(43) Pub. Date: Nov. 27, 2025

(54) MODULAR SYSTEM AND METHODS FOR VACUUM VAPORIZATION FOR COUPLING AND ANALYSIS IN GAS CHROMATOGRAPH APPLICABLE TO HYDROCARBONS PRESENT IN OIL SOURCE ROCKS

(71) Applicant: PETRÓLEO BRASILEIRO S.A. – PETROBRAS, Rio de Janeiro (BR)

(72) Inventors: Rodrigo CABRAL DA SILVA, Rio de Janeiro (BR); Ricardo de Oliveira BACELAR, Rio de Janeiro (BR)

(21) Appl. No.: 19/195,996

(22) Filed: May 1, 2025

(30) Foreign Application Priority Data
May 22, 2024 (BR) 1020240101502

(51) Int. Cl. G01N 30/12; B01D 53/02; G01N 30/06; G01N 30/30; G01N 33/24

(52) U.S. Cl. CPC G01N 30/12; B01D 53/02; G01N 30/30; G01N 33/241; B01D 2257/702; G01N 2030/062; G01N 2030/126

(57) ABSTRACT

The present disclosure relates to embodiments of a systems and methods including a rock sample loading cell, with submission to vacuum and temperature control. A system allows the direct chromatographic analysis of the rocks, without the need for early extraction and sample preparation, which reduces the cost of analysis time and solvents, and favors less generation of residue. Additionally, by means of an integrated vacuum system, it promotes the elimination of air and the increase in the detection of light components from C3 to C10, previously eliminated by the solvent evaporation processes applied in the conventional analysis methods that use early extraction of the rock. The system can be applied to any valved gas chromatography equipment with a gas injection loop, with flame ionization detection (FID) or even a mass spectrometer.

### FIELD OF THE DISCLOSURE

The present disclosure falls within the technical field of oil and gas, specifically related to the modeling, simulation and evaluation of oil reservoirs, and refers to a modular system for vacuum vaporization of hydrocarbons present in oil source rocks, for coupling and analysis in a gas chromatograph.

### BACKGROUNDS OF THE DISCLOSURE

The compositional analysis of hydrocarbons present in source rocks is of great importance in the study and understanding of the oil-rock correlations in oil systems.

In gas chromatographic systems, the lack of devices or systems that allow the direct analysis of hydrocarbons and other volatile compounds present in these source rocks requires additional sample preparation techniques, such as extraction of the rock with solvents, fractionation of the extract by liquid chromatography, solid phase extraction (SPE), precipitation of asphaltenes, etc.

In this context, the need for this sample preparation for the compositional characterization of the source rocks increases both the analysis time and the consumption of solvents and, consequently, the analytical cost and the generation of residue.

For compositional analysis of the volatile organic compounds (linear, branched, cyclic and aromatic hydrocarbons) present in source rocks by Gas Chromatography (GC), it is common to previously perform an extraction step of this rock with mixtures of organic solvents, such as dichloromethane and methanol. Due to the low permeability of the source rocks, such an extraction is normally carried out using a Soxhlet extraction device or Accelerated Solvent Extraction (ASE) equipment.

In addition, depending on the complexity of the sample and the configuration of the gas chromatograph where the components are to be read, additional sample preparations may be further required, such as the asphaltene precipitation and fractionation by liquid chromatography, in order to reduce the complexity of the matrix so as not to compromise the useful life of the chromatographic column and the injection system.

The problem with this analytical route is that it requires a large volume of toxic and flammable organic solvents, which demands a considerable analytical time before the effective compositional reading of the organic matrix of interest, in addition to limiting the analytical capacity to read the lighter components (with chains shorter than 15 carbon atoms), due to the high loss of these components during evaporation processes required to reduce the volumes extracted from the analyzed rocks.

In view of the disclosure, in order to solve the limitations and technical problems described above, the present disclosure describes a modular system consisting of a rock sample loading cell, with submission to vacuum and temperature control. The developed modular system allows the direct chromatographic analysis of the rocks, without the need for early extractions and sample preparation, which reduces the cost of analysis time and solvents, and favors the less generation of residue.

### STATE OF THE ART

Document RU2739138C1 is part of the general state of the art and describes a system and a method for analyzing the total and individual content of hydrocarbons in oil rock samples by gas chromatography with sample pyrolysis. In turn, document U.S. Pat. No. 8,569,685B2 is also part of the general state of the art and proposes a portable analytical system for the rapid analysis of fluids on site and a method for using this system at the well site for the analysis of samples extracted from drilling fluids. The system can be deployed at the well site and used to perform detailed real-time analyses of formation fluids and rock cuttings extracts.

In turn, document PI02012235 describes a device and method for sampling gases and vapors from soil (oil source rocks). The document mentions that the direct sampling of soil gases and vapors is ideal, as it does not have the disadvantage of collecting and canning soils for gas and vapor analysis, in addition to presenting additional advantages, such as the possibility of immediate analysis in loco.

Document U.S. Pat. No. 4,629,702A describes a method for classifying kerogenic materials; for example, materials found in raw rocks, sediments and the like, which comprises the pyrolysis of a sample containing said materials, the analysis of the pyrolysis gases by gas chromatography, and the classification of the kerogen by type, that is, oily, carbon, or mixed type, based on proportions of selected hydrocarbons.

Finally, document U.S. Pat. No. 4,824,790A, which is also part of the general state of the art, describes a system for the chemical analysis of coal and other hydrocarbons (oil source rocks), combines the TGA and FT-IR principles, and uses helium to transport the volatiles evolved by the pyrolysis of the sample to the optical cell of the FT-IR spectrometer. The connection between the TGA furnace and the optical cell is direct and does not impede the flow of fluid, and the carrier gas is preheated to the same temperature as the sample, preferably using a common heating element.

In this way, in view of the documents located in the State of the Art, it is noted that the present disclosure differs from the others in a fundamental technical aspect: only the proposed system has a coupled vacuum device. In the aforementioned documents, there is no such solution.

This technical feature allows for a significant difference because, with such an apparatus, there is greater performance and yield in the vaporization process, with a greater concentration of compounds for reading in the coupled gas chromatograph. In addition, it is important to highlight that the modular system is not a pyrolizer, as in the other documents, nor a conventional vaporizer coupled to the GC, but rather a vacuum vaporizer that is coupled to a gas chromatograph.

In this way, considering the disclosure above, it is possible to perceive relevant differences between the solutions presented in the State of the Art in relation to the solution of this disclosure, and it is further possible to verify the presence of a differential technical effect in the present disclosure, considering the intrinsic advantages of the modular system.

It is important to highlight that the present disclosure provides advantages associated with the elimination of the handling of toxic and flammable organic solvents, reducing unhealthy and dangerous occupational exposure, which can cause damage to facilities and workers' absences, interrupting highly urgent analytical services (such as cases related to spilled oils and clandestine diversions). The presented system has a direct impact on health and safety issues, since, on the one hand, it provides a reduction in risks associated with the health of workers due to exposure to toxic products, such as solvent vapors, such as dichloromethane and methanol, and, on the other hand, it minimizes the risk of fires caused by the handling of flammable solvents, such as methanol and hexane.

In addition, the application of the developed system significantly reduces the time required by technicians in the analysis (MH) of the rock extracts by gas chromatography, since it eliminates the extraction and sample preparation steps normally applied when using conventional methods, which do not use the direct analysis of the source rocks.

The application of the developed modular system allows for more robust quantification of the hydrocarbon components present in the analyzed source rocks and a greater scope of information on components not previously reached by conventional analytical routes for characterizing extracts of oil source rocks, since said system also allows for the arrival and detection of lighter components (from C4 to C13) in the chromatographic reading system. This gain in information expands the comparison zone in oil-rock correlations in the geochemical characterizations, resulting in greater reliability in the generated models of oil systems.

### BRIEF DESCRIPTION OF THE DISCLOSURE

The present disclosure relates to embodiments of a modular system including a rock sample loading cell, with vacuum submission and temperature control. Embodiments of systems and methods allow for the direct chromatographic analysis of the rocks, without the need for early extraction and sample preparation, which reduces the cost of analysis time and solvents, in addition to favoring less generation of residue. Additionally, by means of an integrated vacuum device, a system of the disclosure promotes the elimination of air and the increase in the detection of light components from C3 to C10, previously eliminated in the evaporation processes of the solvents applied in conventional analysis methods that use early extraction of the rock.

Embodiments of the systems and methods can be applied to any valved gas chromatography system with a gas injection loop, with flame ionization detection (FID) or even a mass spectrometer.

### BRIEF DESCRIPTION OF THE FIGURES

In order to obtain a complete and complete view of the object of this disclosure, the figures to which references are made below are presented.

FIG. 1 schematically represents the components of an embodiment of system and the gas chromatograph used for coupling according to the disclosure.

FIG. 2 schematically shows an isometric view of a modular system developed for loading rock samples and vaporizing the hydrocarbons present according to an endpoint of the disclosure.

FIG. 3 schematically represents an exploded view of the modular system developed for loading rock samples and vaporizing the hydrocarbons present according to embodiments of the disclosure.

FIG. 4 schematically represents a chromatogram of the result obtained without the modular system of the present disclosure, that is, with the need to extract components from the rock.

FIG. 5 schematically represents a chromatogram of the result obtained using a system, that is, with the direct reading of the hydrocarbons generated by heating the rock within the system, according to an embodiment of the disclosure.

### DETAILED DESCRIPTION OF THE DISCLOSURE

The present disclosure relates to a modular system consisting of a rock sample loading cell, with vacuum submission and temperature control. The developed system allows the direct chromatographic analysis of the rocks, without the need for early extraction and sample preparation, which reduces the cost of analysis time and solvents, in addition to favoring less generation of residue.

In addition, by means of an integrated vacuum device, the system promotes the elimination of air and the increase in the detection of light components from C3 to C10, previously eliminated in the evaporation processes of the solvents applied in conventional analysis methods that use early extraction in the rock.

Additionally, the aforementioned system minimizes the differential loss of hydrocarbons present in relation to the internal analytical standards inserted directly into the analyzed rock, thus allowing the quantification of detected components with greater precision and accuracy, compared to procedures that require early extraction of the rocks. The solvent extraction processes normally result in the differential evaporative loss of light hydrocarbons in relation to the internal standards added to the rock sample and, therefore, reduce the precision and accuracy in the quantification of the hydrocarbons of interest, in the range of C3 to C10.

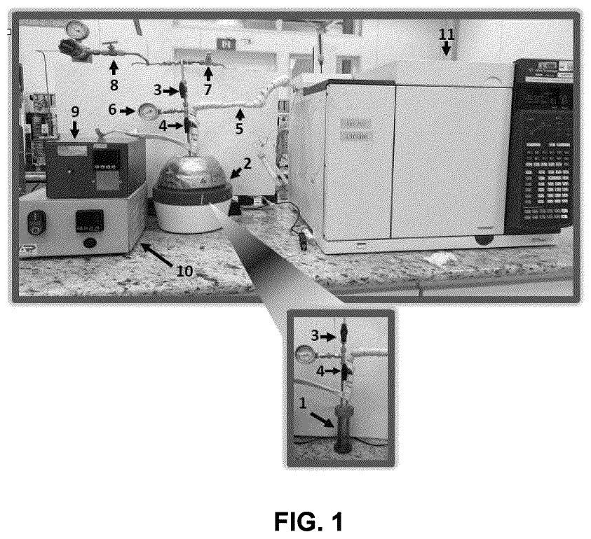

As shown in FIG. 1, the system comprises a rock sample loading and hydrocarbon vaporization cell (1), a temperature controller or indicator (9) of the heating tape or blanket, an outlet valve (4) of hydrocarbon vapors, a temperature controller (10) of the heating blanket of the rock loading cell, a heating blanket (2) of the rock sample loading cell (1), an opening valve (7) of the vacuum system, a dual-scale gauge: pressure gauge and vacuum gauge (6), a inlet valve (3) of vacuum or inert gas in the rock sample loading cell (1), a connection line (5) of the sample loading cell with the GC, an opening valve (8) for inert gas entry.

The heating blanket (2) of the rock sample loading cell (1) comprises a temperature control. The gas from the inlet valve (3) of vacuum or inert gas in the rock sample loading cell (1) is preferably helium gas.

The hydrocarbons from the outlet valve (4) of hydrocarbon vapors are generated by the rock sample for injection into the gas chromatograph (GC). The connection line (5) of the sample loading cell with the GC can be coated with a heating tape or blanket with temperature control.

The temperature controller or indicator (9) of the heating tape or blanket controls the temperature of the heating tape or blanket that covers the connection line of the rock sample loading cell with the GC. The gas chromatograph (11) can be of the valved type with a gas loading loop and with a flame ionization detector (GC-FID).

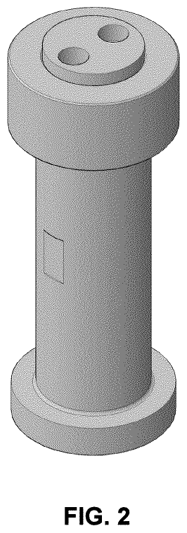

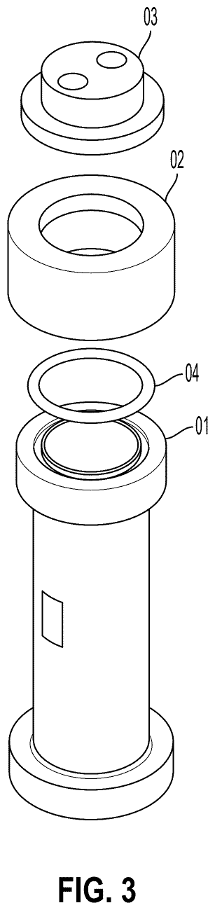

As shown in FIGS. 2 and 3, the rock sample loading and hydrocarbon vaporization cell (1) comprises a body (1.1), a threaded cover (1.2), a closing flange (1.3), and an O-ring (1.4). All these components can be manufactured from AISI 316 steel.

Additionally, as shown in FIG. 1, the rock sample loading and hydrocarbon vaporization cell (1) consists of a pressure gauge (6) with a double scale (pressure gage and vacuum gauge), an inlet valve (3) of vacuum or inert gas into the rock sample loading cell, and an outlet valve (4) of hydrocarbon vapors. The rock sample loading and hydrocarbon vaporization cell (1) is positioned under a thermally insulated heating blanket (2), which covers its body, preventing heat loss during its heating.

The inlet valve (3) of vacuum or inert gas in the rock sample loading cell (1) is connected by a T connection to both the opening valve (7) of the vacuum system and the opening valve (8) for inert gas entry. The outlet valve (4) of hydrocarbon vapors is connected to the line (5) that connects the rock sample loading cell (1) to the gas chromatograph (11). The line (5) is covered with a heating tape/blanket, the temperature of which is controlled by the controller or indicator (9). The heating blanket (2) has its temperature controlled by the controller or indicator (10).

An easy connection as to the developed system is made by coupling the outlet pipe of the same to the gas chromatograph inlet pipe (11).

For full integration between the system and the gas chromatograph (GC), the connection line (5) must be heated to a temperature close to (250° C. to 280° C.) that applied to the rock heating cell (300° C.). In this way, there will be no condensation of analytes inside the pipe and, consequently, all analytes will be transported to the injection loop of the gas chromatograph (GC) without losses that compromise its reading.

This modular system is a closed system, which guarantees the heating of the source rock sample to a maximum of 300° C., with the full vaporization of the hydrocarbons present with chains containing up to at least 10 carbon atoms.

These compounds are detected and identified thanks to the easy coupling of the system developed herein to a gas chromatograph with flame ionization detector (GC-FID) already equipped with a loop system for gas injection.

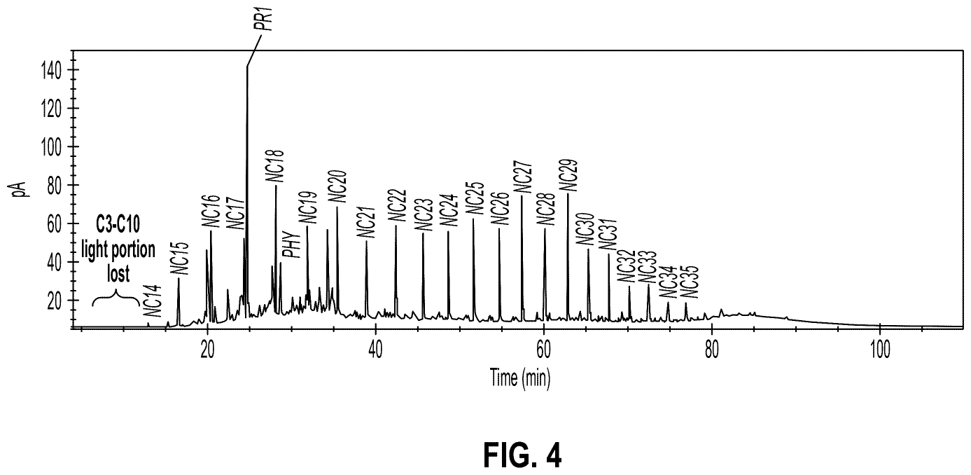

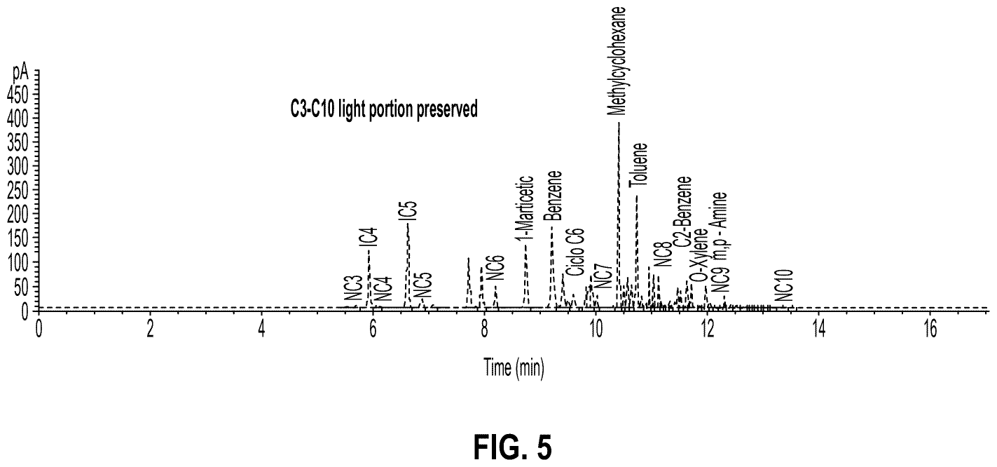

FIGS. 4 and 5 show, respectively, the chromatogram obtained by gas chromatography with flame ionization detection from the extract obtained by the solvent extraction of the rock sample and the chromatogram obtained by direct analysis of this same rock sample by means of the modular vacuum vaporization system coupled to the gas chromatograph. Comparing the two chromatograms, it can be noted that the device developed herein was the only one capable of preserving hydrocarbons from C3 to C10 for detection.

FIG. 4 shows the chromatogram obtained by GC-FID of hydrocarbons present in a sample of rock A (C14-C35), extracted with solvents, where the extract was further subjected to fractionation by liquid chromatography to isolate the portion of saturated hydrocarbons (Conventional Analysis).

FIG. 5 shows the chromatogram obtained by GC-FID of light hydrocarbons from C3 to C10, present in the same sample of rock A, directly subjected to heating in the developed system, coupled to GC-FID.

Those skilled in the art will value the knowledge presented herein and will be able to reproduce the disclosure in the presented embodiments and in other variants, encompassed by the scope of the attached claims.

1. A modular system for vacuum vaporization of hydrocarbons present in oil source rocks, the system comprising:
a rock sample loading and hydrocarbon vaporization cell, a heating blanket of the rock sample loading cell, an inlet valve of vacuum or inert gas in the rock sample loading cell, an outlet valve of hydrocarbon vapors, a connection line of the sample loading cell with the gas chromatograph, a dual-scale gauge: pressure gauge and vacuum gauge, an opening valve of the vacuum system, an opening valve for inert gas entry, a temperature controller or indicator of heating tape or blanket, a temperature controller of the heating blanket of the cell rock loading cell, and a gas chromatograph.

2. The system according to claim 1, wherein the heating blanket of the rock sample loading cell comprises temperature control.

3. The system according to claim 1, wherein the inert gas of the inlet valve of vacuum or inert gas in the rock sample loading cell comprises helium gas.

4. The system according to claim 1, wherein the connection line of the sample loading cell with the gas chromatograph is coated with a heating tape or blanket with temperature control.

5. The system according to claim 1, wherein the gas from the opening valve for gas entry is used for inerting the rock sample loading cell.

6. The system according to claim 1, wherein one or more of the temperature controller, the indicator of the heating tape, or blanket controls the temperature of the heating tape or blanket that covers the connection line of the rock sample loading cell with the gas chromatograph.

7. The system according to claim 1, wherein, the gas chromatograph (11) is of the valved type with a gas loading loop and a flame ionization detector.

8. The system according to claim 1, wherein the rock sample loading and hydrocarbon vaporization cell is positioned under a thermally insulated heating blanket that covers its body, preventing heat loss during its heating.

9. The system according to claim 1, wherein the inlet valve of vacuum or inert gas in the rock sample loading cell is connected by a T connection to both the opening valve of the vacuum system and the opening valve for inert gas entry.

10. The system according to claim 1, wherein the outlet valve of hydrocarbon vapors is connected to the connection line of the rock sample loading cell with the gas chromatograph.

11. The system according to claim 1, wherein the connection line of the rock sample loading cell with the gas chromatograph connects the rock sample loading and hydrocarbon vaporization cell with the gas chromatograph.

12. The system according to claim 1, wherein the connection line of the sample loading cell with the gas chromatograph is covered with a heating tape or blanket to control its temperature by the controller or indicator.

13. The system according to claim 1, wherein the heating blanket has its temperature controlled by the controller or indicator.

14. The system according to claim 1, wherein the source rock sample is heated to a maximum of 300° C.

15. The system according to claim 1, wherein the hydrocarbons comprise chains containing up to at least 10 carbon atoms.
